(19) United States
(12) Patent Application Publication
(10) Pub. No.: US 2024/0092895 A1
(43) Pub. Date: Mar. 21, 2024

(54) CHIMERIC NANOBODY COMPOSITIONS AND METHODS OF TREATMENT THEREOF

(71) Applicant: THE TRUSTEES OF COLUMBIA UNIVERSITY IN THE CITY OF NEW YORK, New York, NY (US)

(72) Inventors: Travis MORGENSTERN, New York, NY (US); Henry COLECRAFT, Robbinsville, NJ (US)

(21) Appl. No.: 18/514,836

(22) Filed: Nov. 20, 2023

Related U.S. Application Data
(63) Continuation of application No. PCT/US2022/030186, filed on May 20, 2022.
(60) Provisional application No. 63/191,582, filed on May 21, 2021.

(51) Int. Cl. C07K 16/28; C07K 16/40

(52) U.S. Cl. CPC C07K 16/28; C07K 16/40; C07K 2317/31; C07K 2317/35; C07K 2317/569; C07K 2317/76

(57) ABSTRACT

The present disclosure provides, inter alia, chimeric nanobody compositions that selectively modulate the function of a target protein within a defined population of cells. Also provided are methods for treating diseases such as ion channelopathies.

### CROSS REFERENCE TO RELATED APPLICATIONS

The present application is a continuation of PCT international application no. PCT/US2022/030186, filed on May 20, 2022, which claims benefit of U.S. Provisional Patent Application Ser. No. 63/191,582, filed on May 21, 2021, which applications are incorporated by reference herein in their entireties.

### GOVERNMENT FUNDING

This invention was made with government support under grant nos. GM107585, DK118866 and HL142111, awarded by National Institutes of Health. The government has certain rights in the invention.

### FIELD OF DISCLOSURE

The present disclosure provides, inter alia, chimeric nanobody compositions and methods for treating diseases such as ion channelopathies.

### INCORPORATION BY REFERENCE OF SEQUENCE LISTING

This application contains references to amino acids and/or nucleic acid sequences that have been filed concurrently herewith as sequence listing XML file “CU21331-seq.xml”, file size of 8,814 bytes, created on Nov. 10, 2023. The aforementioned sequence listing is hereby incorporated by reference in its entirety pursuant to 37 C.F.R. § 1.52(e)(5).

### BACKGROUND OF THE DISCLOSURE

Ubiquitin (Ub) is a small, 9 kilodalton protein that is covalently attached to a target protein as a post-translational modification. The transfer of cytosolic Ub onto a protein is a multi-step reaction that culminates with a class of enzymes called E3 ubiquitin ligases mediating the final step of Ub attachment. There are more than 600 E3 ubiquitin ligases in the human genome; this diversity could in part be explained by the diverse nature of the ubiquitin code: Ub chains are formed by the subsequent addition of Ub onto an existing Ub bound to the target protein. Complexity arises as Ub itself has 7 lysine residues that can be potentially ubiquitinated (as well as its N-terminal methionine); the shape of the resulting polyubiquitin chains can alter the downstream fate of the target protein. For example, K48 E3 ligases have been demonstrated in vitro to add Ub onto the 48th residue of an existing Ub, forming K48 polyubiquitin chains that have been characterized as a signal for proteasomal degradation of the target protein. Additional signaling roles for ubiquitin have been characterized such as K63 branches coordinating endocytosis of plasma membrane proteins. In particular, the HECT family ligase Nedd4-2 has been well characterized to target to the plasma membrane, where it mediates endocytosis and ultimately lysosomal degradation of a host of PY-containing transmembrane proteins such as ENaC, KCNQ1, and Nav1.5.

Targeted ubiquitination with proteolysis-targeting chimeras (PROTACS) small molecules has emerged as an exciting new therapeutic strategy since its inception twenty years ago. Indeed, more than 20 molecules that share a similar mechanism of action are expected to be in clinical use by the end of 2021. A PROTAC is composed of a heterobifunctional small molecule: one end of the molecule binds to a target protein, while the other end of the molecule (connected via a linker) binds to an E3 ubiquitin ligase. The technology redirects a particular E3 ubiquitin ligase to a target protein, resulting in its proteasomal degradation. While PROTACS have been developed towards several targets, mostly in the cancer field, there has yet to be a demonstration of PROTACS applied to integral membrane proteins—a family of proteins that includes more than 50% of current FDA-approved pharmaceutical targets such as ion channels and GPCRs.

There are more than 600 E3 ubiquitin ligases in the human genome, yet thus far PROTACS have been developed to utilize less than a dozen E3 ligases. There are two general types of E3 ubiquitin ligases that differ in respect to the mechanism by which they transfer ubiquitin to the target protein: RING and HECT domain E3 ligases. The RING family comprise the majority of E3 ligases, and have been successfully incorporated into the PROTACS strategy, while the HECT domain ligases have not been successfully recruited with small molecules in the PROTACS format. As known regulators of ion channels, GPCRs, transporters, and kinases, the HECT domain E3 ubiquitin ligases could provide a means to target these proteins for ubiquitination, and therefore hold immense therapeutic potential.

### SUMMARY OF THE DISCLOSURE

The present disclosure describes a novel strategy to redirect cellular Nedd4L, a HECT E3 ubiquitin ligase, to a target protein using genetically-encoded chimeric divalent nanobodies (Divas). A Diva is composed of two nanobodies: one nanobody is specific for the target protein and the other nanobody is specific for the HECT domain of Nedd4L. Importantly, we have a nanobody that is able to recruit an active Nedd4L to a target protein, resulting in ubiquitination and functional inhibition of the target protein. This tool (Diva_{INHIBIT}) has been applied to regulate three different ion channels: the voltage-gated calcium channel, Caᵥ2.2, the voltage-gated potassium channel, KCNQ1, and the epithelial sodium channel, ENaC. Further, there are point mutations in ENaC that prevent Nedd4L recognition of ENaC and cause an early onset hypertension Liddle syndrome; it has been shown in heterologous systems that Diva_{INHIBIT} is capable of restoring Nedd4L regulation of ENaC.

The present disclosure also provides isolated nanobodies that can block the activity of Nedd4L. The activity of Nedd4L on a membrane protein typically results in internalization of the protein from the surface of the cell. Thus, Divas that are able to selectively block the action of Nedd4L on a target protein (Diva_{RESCUE}) can provide a means for selectively upregulating the surface expression of a particular membrane protein. We have shown the ability of Diva_{RESCUE} to strongly upregulate surface levels of the potassium channel KCNQ1. This could have therapeutic relevance for certain ion channelopathies which harbor mutations that prevent proper surface levels of the ion channel, such as subsets of Long QT syndrome and CFTR.

Most promising data showing the potential of Divas as a gene therapy utilizes a proprietary nanobody that targets the auxiliary Caᵥβ subunit of high voltage activated calcium channels (HVACCs). This Caᵥβ was incorporated into the Diva_{INHIBIT} module to create a tool with the potential to target any high voltage activated calcium channel (which all associate with a Caᵥβ subunit). These channels are critical for the function of excitable cells such as neurons, and muscle. As such, they have been prominent drug targets for diseases as diverse as hypertension, cardiac arrhythmias, diabetes, Parkinson's disease, and chronic pain. The latter is estimated to affect 20% of the global population, with large economic and social consequences. Despite this large prevalence, current treatments for chronic pain remain inadequate, as exemplified with a common treatment for chronic pain that has led to an epidemic: opioids.

Among HVACCs, the N-type calcium channel is a primary target for the treatment of chronic pain. It is well established that these channels are expressed in the sensory neurons that transmit the sensation of pain, that blockade of these channels can disrupt pain perception, and that these channels are upregulated in multiple models of pain. An N-type calcium channel blocker derived from marine snail venom (Prialt) is currently used in limited situations for pain management, but is limited by a narrow therapeutic window. The therapeutic potential to decrease/inhibit HVACCs in particular tissues extend beyond pain: calcium channel blockers are currently in clinical trials for the treatment of diabetes as well as Parkinson's disease. All current drugs block the channel at its functional location, the plasma membrane, by disrupting its ability to permit calcium entry into the cell. Given their central importance in many cells, a primary concern with all current pharmacological HVACC blockers is off-target effects. The present disclosure suggests a strategy to genetically encode HVACC inhibitors, providing a means to spatially restrict therapy to a certain tissue or cell population.

Most drugs against HVACCs target the pore-forming α₁ subunit of the channel, disrupting the ability of the channel to permit calcium entry into the cell. HVACCs are also composed of auxiliary β and α₂-δ subunits which function to facilitate trafficking of the α₁ subunit to the plasma membrane and fine-tune its properties once there. As critical regulators of channel function, these auxiliary subunits are potential therapeutic targets. Indeed, Gabapentin (Pregabalin or Lyrica), is used for treatment of chronic pain and works by downregulating N-type calcium channels via an association with the α₂-δ subunit of the channel (the exact mechanism of how this occurs is under intense study). Gabapentin-based treatment produces fewer side effects then other treatment options such as opioids or tricyclic antidepressants, but full alleviation of pain is rare. Thus, there is a large need for improved treatment options for this common condition.

The auxiliary β subunit is obligatory for proper channel trafficking. Yet, despite efforts to target the α₁-β binding interface, this subunit remains an untapped therapeutic target. Not wishing to be bound by a particular theory, it is believed that our technology can simultaneously target the β subunit and the E3 ligase Nedd4L (which is expressed in pain-sensing neurons) to take advantage of the ubiquitin pathway to remove HVACCs from the cell surface. Given the diverse physiological functions of HVACCs, our technology could be applied for research or therapeutic purposes in numerous fields. Herein we focus, but are not limited to, the application of our technology for the study and treatment of chronic pain.

The present disclosure provides a tool that is comprised of a series of novel nanobodies that are able to bind Nedd4L and either permit or block its activity, which are targeted to a particular protein using an additional nanobody towards the target. The tool uses a fundamentally different strategy than all current calcium channel blockers: rather than physically blocking the channel from performing its function, the channel is removed from its functional destination, the plasma membrane. Further, this is accomplished in a genetically encoded manner, allowing cellular specificity to be achieved with, but not limited to, transfection or viral vector-based methods. Further, it has been shown that the general Diva strategy is applicable to three different ion channels, suggesting that the strategy may be employed to target a variety of membrane proteins.

One aspect of the present disclosure is directed to the creation of genetically encoded chimeric divalent nanobodies (Divas). Accordingly, one embodiment of the present disclosure is a chimeric divalent molecule. The molecule comprises: an E3 ubiquitin ligase binder, a target binder, and a variable linker between the E3 ubiquitin ligase binder and the target binder.

Another embodiment of the present disclosure is a method for treating or ameliorating the effects of a disease in a subject. The method comprises administering to the subject an effective amount of a chimeric divalent molecule disclosed herein.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic showing PROTACS-inspired Diva design.

FIGS. 2A-2G show that Divaβ-GFP is able to reduce surface density of reconstituted Caᵥ2.2 channels. FIG. 2A shows the Diva design. FIG. 2B shows the Modular domains of Nedd4L. FIG. 2C is a schematic of experimental strategy. In FIGS. 2D and 2E, above, cartoons depicting experimental condition and below, exemplar flow cytometry contour plots of cells expressing BBS-α1B, YFP-β2a, α2δ-1, YFP, and either βnb+GFPnb (not tethered) (FIG. 2D) or Divaβ-GFP (FIG. 2E). FIGS. 2F and 2G are same as FIGS. 2D and 2E, respectively, but with YFP-HECT_{NEDD}4L transfected in place of YFP.

FIGS. 3A-3D show the generation of HECT_{Nedd4L} nanobodies. In FIG. 3A, above, domains of Nedd4L. Below: Left, Coomassie gel of purified HECT_{Nedd4L}. Right, crystal structure of HECT_{Nedd4L} (PDB: 3JW0). FIG. 3B shows the yeast-display selection scheme. FIG. 3C is a cartoon of yeast showing epitope tags and antibodies used for labeling during selection. FIG. 3D shows Exemplar data of clonal isolation using FACS and flow cytometry assessment of clonal binding.

FIGS. 4A-4C show that HECT_{Nedd4L} nanobodies bind Nedd4L within the cytosol of mammalian cells, with exemplar flow-FRET binding curves. Candidate cerulean tagged-nanobodies were co-expressed in HEK293 cells with venus-HECT_{Nedd4L} (FIG. 4A), venus-full length-Nedd4L (FIG. 4B), or venus-HECT_{Nedd4-1} (FIG. 4C).

FIGS. 5A-5F show the functional impact of Divaβ-Nedd4L on reconstituted Caᵥ2.2 channels. FIG. 5A is the schematic of experimental design. FIG. 5B shows exemplar contour plots of flow cytometry experiments in HEK293 cells transfected according to FIG. 5A with or without overexpression of Nedd4L. FIGS. 5C and 5D are summary data of surface (FIG. 5C) and total Caᵥβ (FIG. 5D). Each data set was normalized to control group that expressed CFP. n>3,000 cells analyzed per experiment, N>2 separate experiments, s.e.m. * P<0.001 compared with other conditions, one-way ANOVA with Tukey's multiple comparison test. FIG. 5E shows exemplar traces (top) and summary J-V curves (bottom) from whole-cell patch clamp measurements in HEK293 cells expressing α_{1B}, β₂ₐ, α₂δ, and βnb (squares) or Divaβ-C11 (triangles). FIG. 5F is in the same format as FIG. 5E, but with HEK cells in which Nedd4L was also over-expressed.

FIGS. 6A-6D show that Divaβ-C11 reduces HVACC in cultured DRG neurons. FIG. 6A is schematic of adenoviral construct encoding bicistronic expression of tdTomato and Diva_{β-C11}. FIG. 6B shows confocal image of tdTomato fluorescence from infected DRG neurons expressing Divaβ-C11. FIG. 6C shows immunofluorescence of Nedd4L staining from murine L4 DRG. FIG. 6D shows exemplar (top) and summary J-V curves (bottom) from whole-cell patch clamp measurements from DRG neurons infected with mCherry (circles) or Divaβ-C11 (squares). *P<0.001, unpaired two-tailed Student's t-test.

FIG. 7A is a schematic showing that the epithelial sodium channel (ENaC) is regulated by Nedd4L.

FIG. 7B shows that mutations in the PY motif of ENaC cause Liddle syndrome.

FIGS. 8A-8D show that DivaGFP-C11 reduces HVACC in cultured DRG neurons. FIG. 8A is schematic of experimental design using DivaGFP-C11. FIG. 8B shows exemplar (top) and J-V population curves (bottom) from whole-cell measurements of HEK293 cells expressing wildtype ENaC and GFPnb (circels) or DivaGFP-C11 (squares). FIG. 8C is sequence alignment of PY motif found in β-ENaC. FIG. 8D shows whole-cell measurements as in FIG. 8B, using the mutant P617 β-ENaC.

### DETAILED DESCRIPTION OF THE DISCLOSURE

High voltage-activated calcium channels (HVACCs) mediate many key cellular processes of excitable cells including muscle contraction, neurotransmitter secretion, and gene regulation (Catterall, 2000). These channels exist as a multiprotein complex composed of one of seven transmembrane α₁ subunits (α_{1A}-α_{1F}; α_{1S}), one of four possible cytosolic β subunits (β₁-β₄), and one of four possible transmembrane α₂δ subunits (α₂δ-1-α₂δ-4). The α₁ subunit contains the pore and voltage-sensor, while the auxiliary β and α₂δ subunits facilitate trafficking of the α₁ subunit to the plasma membrane and modulate gating of the channel (Catterall, 2000; Dolphin, 2012). HVACCs are prominent and potential therapeutic targets for a variety of diseases including hypertension, cardiac arrhythmia, pain, diabetes, and Parkinson's disease (Zamponi et al. 2015). Current pharmacological approaches target the α₁ or α₂δ with small molecules that block calcium permeation, movement of the voltage-sensors, or critical protein-protein interactions (Zamponi et al. 2015). Yet upon administration, these small molecules are widely distributed within the tissues of the body and often cause off-target effects due to blockade of non-pathological calcium channels (Schmidtko et al. 2010). In certain conditions genetically encoded calcium channel inhibitors (GECCIs) may be advantageous over small molecules, as they could potentially provide long-lasting blockade with enhanced tissue specificity (Xu et al. 2009).

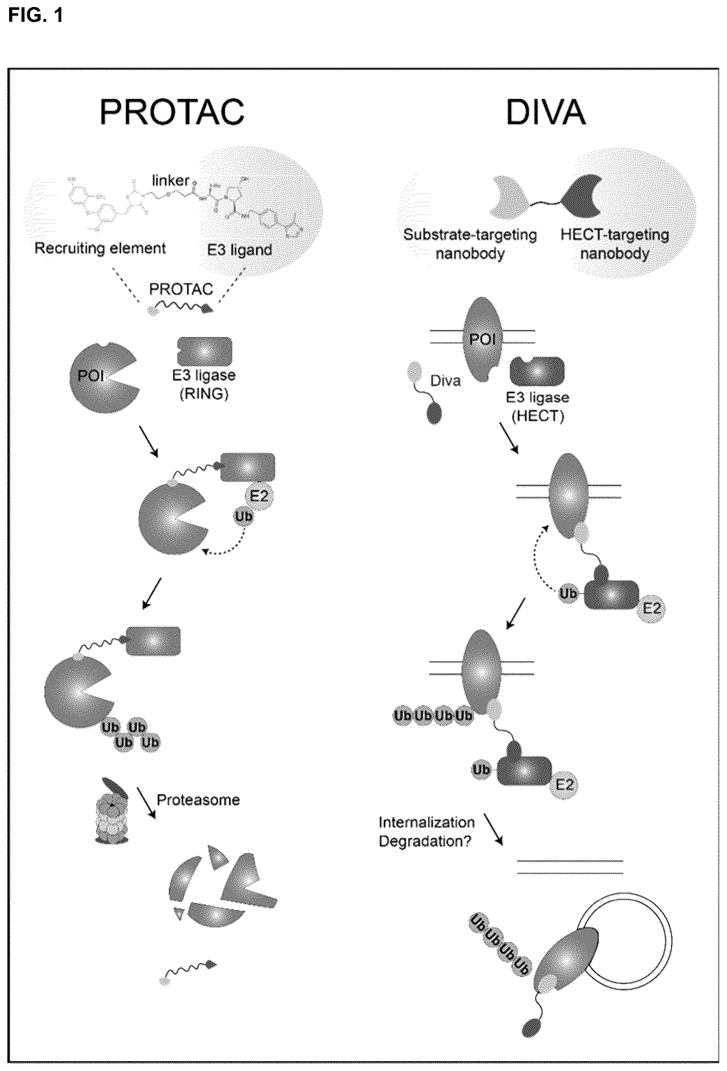

Our previous work created a potent GECCI by pairing the specificity of camelid single-domain antibodies (herein referred to as nanobodies) towards the cytosolic Caᵥβ of HVACCs with the catalytic activity of the HECT family E3 ubiquitin ligase Nedd4L (also known as Nedd4-2), to achieve targeted ubiquitination and functional inhibition of the channel (Morgenstern et al. 2019). Yet ubiquitination is a powerful post-translational modification, and dysregulation of ubiquitination has been implicated in cancer, neurodegenerative diseases, and viral budding (MacGurn et al. 2012; Yau et al. 2017; Hicke, 2001). The cell maintains ubiquitin homeostasis in part thru regulation of ubiquitin E3 ligases: the HECT ligases are subject to several layers of regulation, including autoinhibition (Wiesner et al. 2007; Mari et al. 2014; Chen, 2017), phosphorylation (Debonneville et al. 2001), and sequestration by scaffolding proteins (Hayer and Bhalla, 2005). This high degree of regulation suggests that overexpression of an unregulated catalytic subunit may lead to off-target ubiquitination, with potentially deleterious consequences. In an attempt to circumvent this concern, we adopted an approach inspired by proteolysis-targeting chimeras (PROTACs) (Lai et al. 2016) in which we hypothesized that divalent nanobodies could simultaneously bind a protein of interest and endogenous Nedd4L, resulting in targeted ubiquitination (FIG. 1). Here, we isolate nanobodies towards the HECT domain of Nedd4L and create Divas: divalent nanobodies composed of a substrate-targeting nanobody tethered to a HECT-targeting nanobody. We use a Caᵥβ nanobody to target Divas to HVACCs and identify a HECT_{Nedd4L} nanobody that is able to inhibit HVACC in heterologous systems as well as dorsal root ganglion neurons. Lastly, show that Divas can achieve targeted ubiquitination of an established Nedd4L substrate, the sodium epithelial channel, ENaC and further, restore Nedd4L modulation of a mutant ENaC that is unable to bind Nedd4L. Divas thus represent a novel class of genetically encoded blockers that can be used to target multiple ion channels for in vivo and potential therapeutic applications. Further, to our knowledge, this is the first evidence of an endogenous HECT domain ubiquitin ligases being manipulated for targeted ubiquitination (Ottis et al. 2017), a finding that could have broad implications for the burgeoning field of PROTACs.

Accordingly, one embodiment of the present disclosure is a chimeric divalent molecule. The molecule comprises: an E3 ubiquitin ligase binder, a target binder, and a variable linker between the E3 ubiquitin ligase binder and the target binder.

In some embodiments, the E3 ubiquitin ligase binder and the target binder are both nanobodies. As used herein, the term “nanobodies” means small antibody fragments derived from a class of camelid antibodies having a small size ( 1/10th the size of a conventional antibody).

In some embodiments, the E3 ubiquitin ligase is a HECT domain E3 ubiquitin ligase. In some embodiments, the HECT domain E3 ubiquitin ligase is Nedd4L.

In some embodiments, the E3 ubiquitin ligase binder recruits an active Nedd4L to the target. In some embodiments, the E3 ubiquitin ligase binder selectively blocks the action of Nedd4L on the target. As used herein, the term “block” means partially or completely interfering with a protein of interest so as to achieve a desired clinical effect.

In some embodiments, the target is a protein selected from ion channels, G protein-coupled receptors (GPCRs), cystic fibrosis transmembrane conductance regulator (CFTR), transporters, and kinases.

In some embodiments, the target is an ion channel selected from the voltage-gated calcium channel, CaV2.2, the voltage-gated potassium channel, KCNQ1, and the epithelial sodium channel, ENaC.

In some embodiments, the target is a high voltage-activated calcium channel (HVACC). In some embodiments, the HVACC is an N-type calcium channel. In some embodiments, the target is the auxiliary CaVβ subunit of the HVACC.

Another embodiment of the present disclosure is a method for treating or ameliorating the effects of a disease in a subject. The method comprises administering to the subject an effective amount of a chimeric divalent molecule disclosed herein.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

In the present disclosure, an “effective amount” or “therapeutically effective amount” of a composition is an amount of such a composition that is sufficient to effect beneficial or desired results as described herein when administered to a subject or contacted with a cell. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a composition according to the disclosure will be that amount of the composition, which is the lowest dose effective to produce the desired effect with no or minimal side effects. The effective dose of a composition according to the present disclosure may be administered as two, three, four, five, six or more sub-doses, administered separately at appropriate intervals throughout the day.

In some embodiments, the disease is selected from the group consisting of an inherited ion channelopathy, a cancer, a cardiovascular condition, an infectious disease, and a metabolic disease. Non-limiting examples of an inherited ion channelopathy include epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus.

In some embodiments, the disease is selected from the group consisting of hypertension, Liddle syndrome, cardiac arrhythmias, diabetes, Parkinson's disease, and chronic pain. In some embodiments, the disease is chronic pain.

### EXAMPLES

The following examples are provided to further illustrate certain aspects of the present disclosure. These examples are illustrative only and are not intended to limit the scope of the disclosure in any way.

### Example 1

### Inhibition of Multiple Ion Channels with Divalent Nanobodies that Recruit Nedd4L for Targeted Ubiquitination

### Results

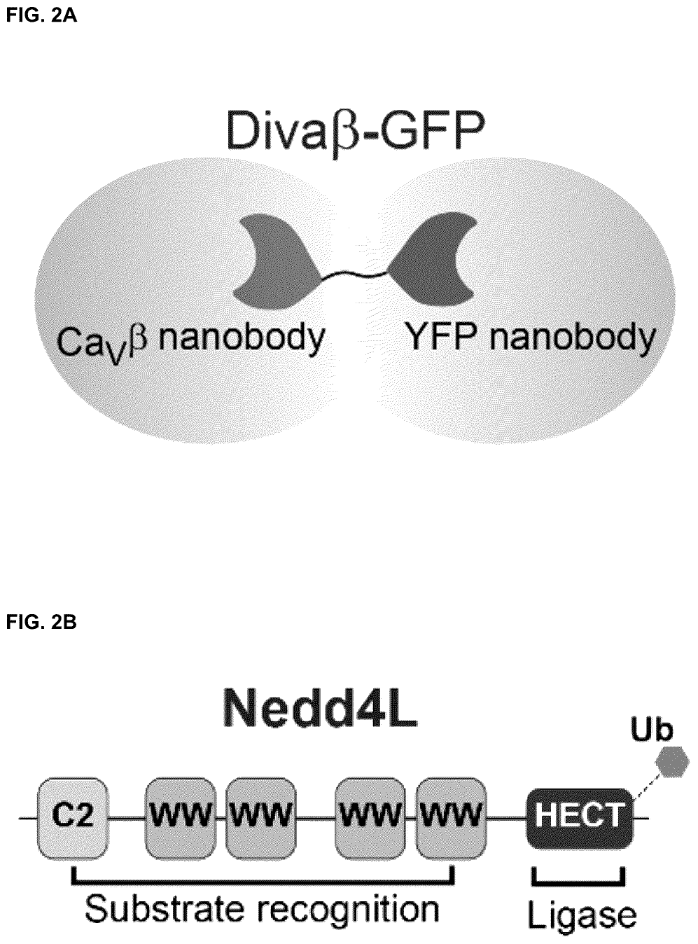

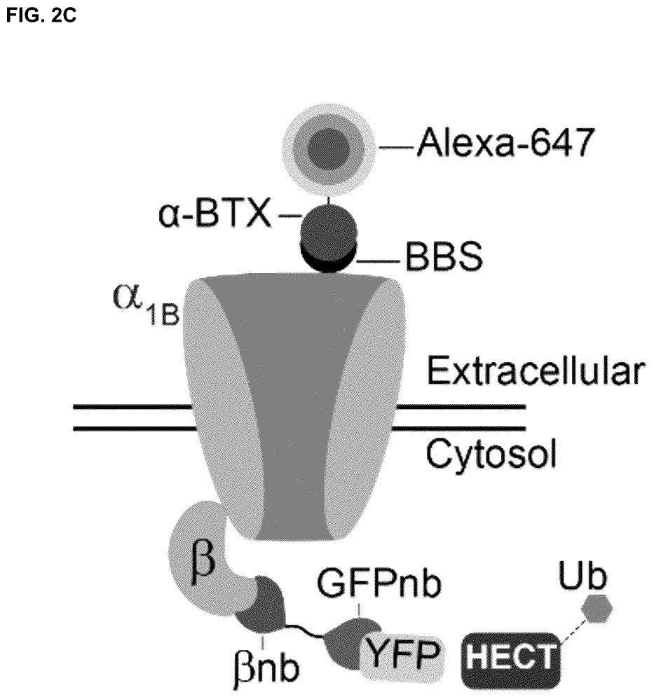

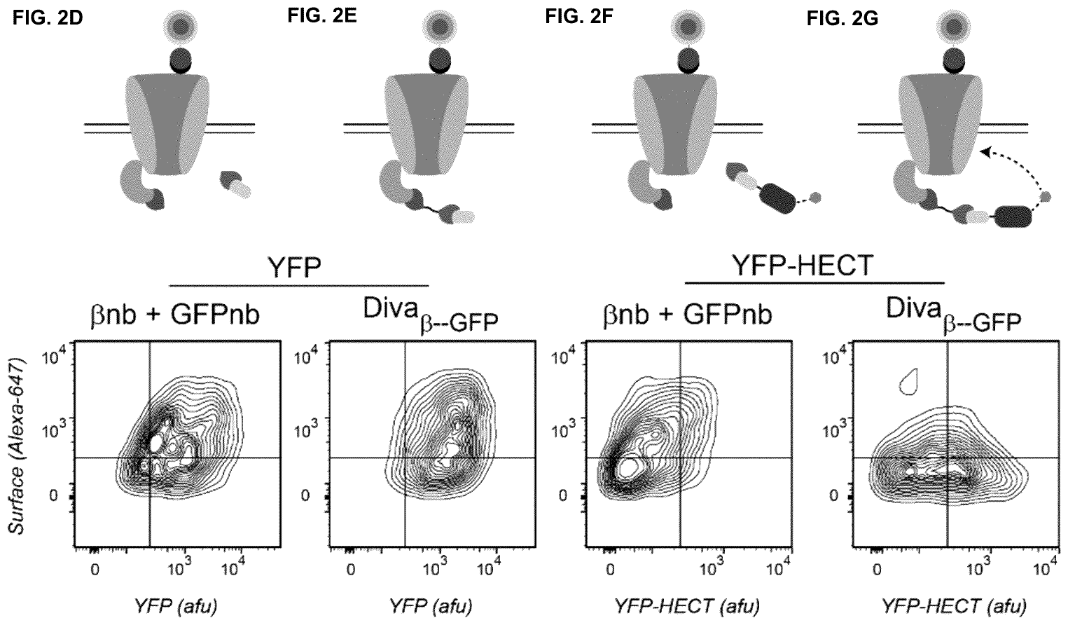

A chimeric Caᵥβ nanobody (βnb) fused to the catalytic HECT domain of Nedd4L (HECT_{Nedd4L}) was used previously to achieve targeted ubiquitination and elimination of functional HVACCs from the surface. Here, we reasoned that if we were able to use a nanobody to recruit HECT_{Nedd4L} to a protein of interest, we would be able to achieve similar regulation. To test this hypothesis, we created a heterobifunctional molecule comprised of βnb, a (GGGGS)₂ linker, and a nanobody that binds GFP/YFP, but not CFP (GFPnb) (Kubala et al. 2010) (FIG. 2A), and tested its ability to recruit a YFP-tagged HECT_{Nedd4L} domain to regulate the calcium channel. We call this general approach that utilizes divalent nanobodies Diva, and this particular construct Divaβ-GFP. We used flow cytometry to measure the potential effects of Divaβ-GFP on surface levels of Caᵥ2.2 in HEK293 cells; the assay features an α_{1B} subunit that harbors a tandem 13-residue bungarotoxin-binding site (BBS) in the extracellular S5-S6 loop of domain IV, permitting selective labeling of surface channels in living cells with the non-permeable bungarotoxin-conjugated Alexa-647 dye (Morgenstern and Colecraft, 2021) (FIG. 2C). Baseline measurements of control cells that expressed the inert βnb and GFPnb separately (not physically linked) along with BBS-α_{1B}, β₂ₐ, α₂δ-1 and YFP showed a population of cells that had robust surface Caᵥ2.2 fluorescence (FIG. 2D). There was no change in surface fluorescence when Divaβ-GFP was coexpressed with YFP and the same channel subunits, establishing that Divaβ-GFP had no basal effect on channel surface density (FIG. 2E). We then tested the same conditions in cells co-transfected YFP-HECT_{Nedd4L}. Cells which expressed βnb+GFPnb with YFP-HECT_{Nedd4L} had no discernible effect on surface density of BBS-Caᵥ2.2, although YFP expression was markedly lower, possibly due to autoubiquitination of the HECT domain (FIG. 2F) (Chen, 2017). In contrast, when Divaβ-GFP was co-expressed with YFP-HECT_{Nedd4L} there was a striking reduction in surface density of Caᵥ2.2 (FIG. 2G). Excitingly, this result was similar to what we had observed previously with our pnb-HECT_{Nedd4L} fusion (Morgenstern et al. 2019) and suggested that nanobody-mediated recruitment of Nedd4L domain may be a viable strategy to regulate target proteins.

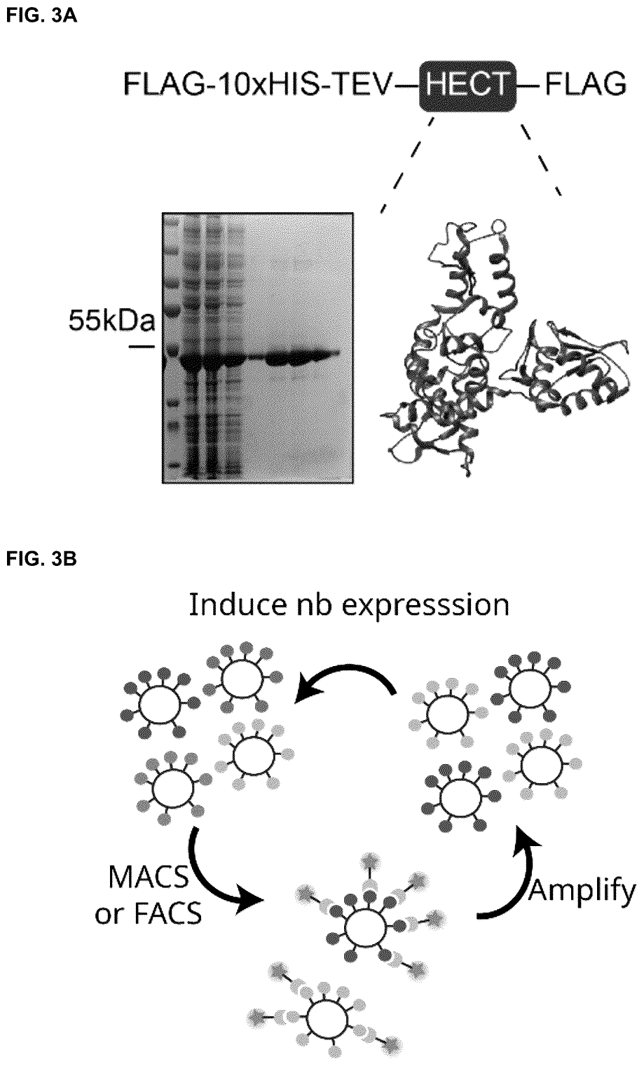

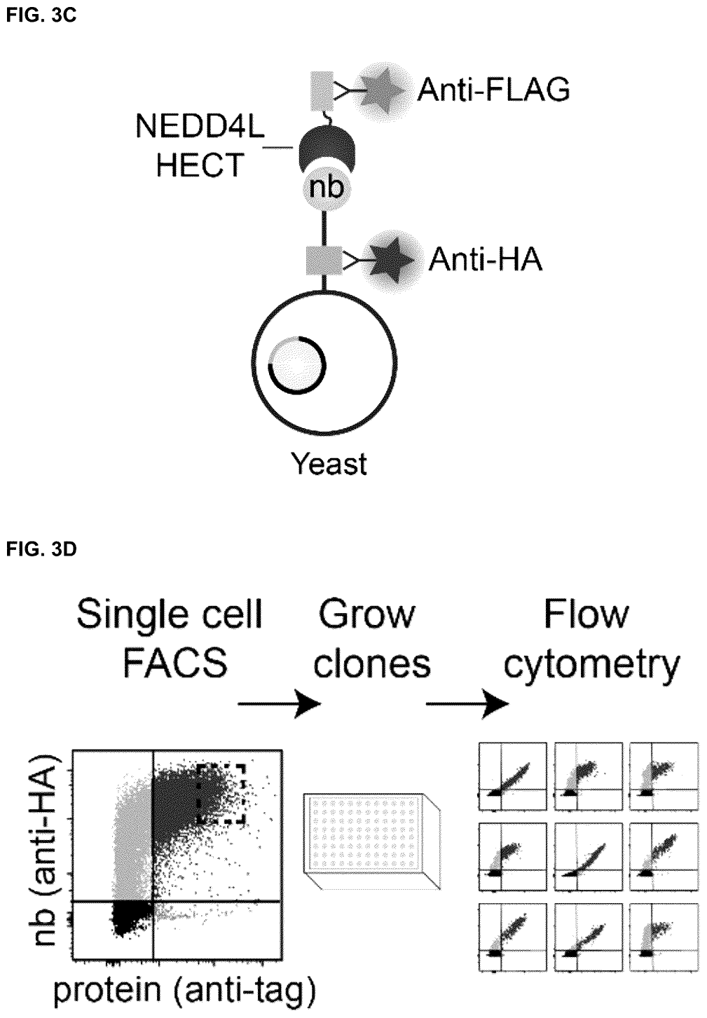

Encouraged by the results above, we set out to generate nanobodies towards HECT_{Nedd4L}. To do so, we first purified the catalytic HECT domain of Nedd4L (corresponding to residues 596-975 of full-length Nedd4L) bearing an N and C-terminus 10× His tag and FLAG tags, respectively, from E. coli using immobilized metal affinity chromatography (FIG. 3A). We used this purified HECT_{Nedd4L} as bait to isolate nanboodies using yeast display from a library of yeast that express 1×10⁸ unique, synthetic nanobodies on their extracellular surface (McMahon et al. 2018) (FIGS. 3B and 3C). We selected for nanobodies using 1 μM HECT_{Nedd4L} as bait and performed two sequential rounds of magnetic-activated cell sorting using. We then performed fluorescence-activated cell sorting to isolate individual clones into a 96-well plate and assessed their capacity to bind HECT_{Nedd4L} with flow cytometry (FIG. 3D).

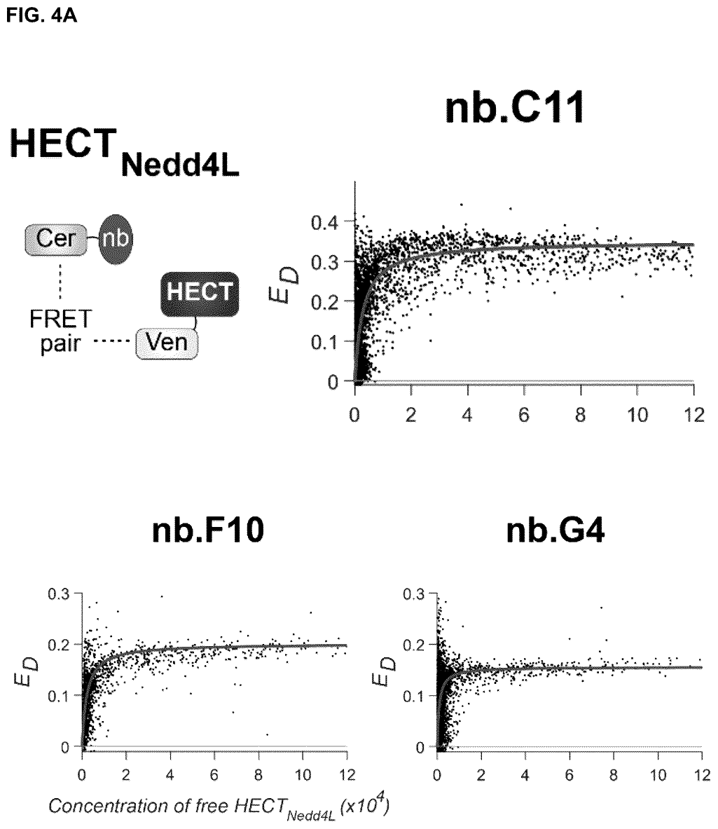

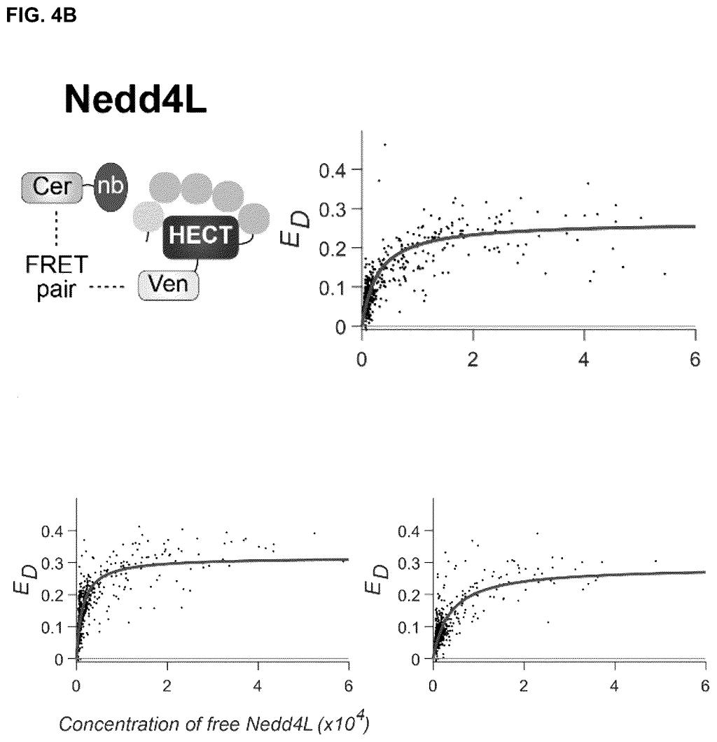

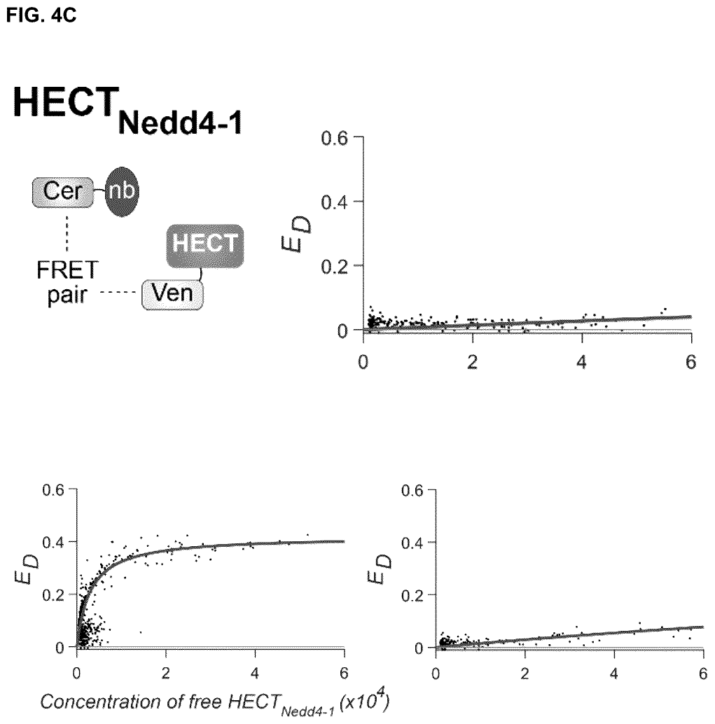

Promising candidates that showed strong binding in the on-yeast format were cloned into a mammalian expression vector with an in-frame cerulean tag and co-transfected in HEK293 cells with venus-HECT_{Nedd4L} for flow-cytometry based Förster resonance energy transfer (Flow-FRET) experiments (Rivas et al. 2021). As shown in FIG. 4A, a number of nanobodies (but not all candidates) retained their ability to bind HECT_{Nedd4L} within the reducing environment of the cytosol. This was promising, but reports of autoinhibititon of full-length-Nedd4L via intramolecular interactions between the HECT and C2 (Mari et al. 2014; Wiesner et al. 2007) or WW domains (Bruce et al. 2008) led us to question whether the epitopes for these nanobodies would be accessible in the full-length protein. To address this, we repeated our flow-FRET experiment using full-length Venus-Nedd4L with each nanobody (FIG. 4B) and observed that three previously identified high-affinity nanobodies were able to also bind the full-length Nedd4L (FIG. 4B). Lastly, we addressed the question of specificity: Nedd4-1 is the closest homolog of Nedd4L, with 82% amino acid sequence conserved between their HECT domains, yet we observed no detectable binding between Venus-HECT_{Nedd4-1} and two of the three candidate nanobodies (nb.C11 and nb.G4) using our flow-FRET assay (FIG. 4D). These experiments verified that the HECT_{Nedd4L} nanobodies are capable of binding full-length Nedd4L within the cytosol of a mammalian cell, and so we next incorporated them into our Diva-based strategy.

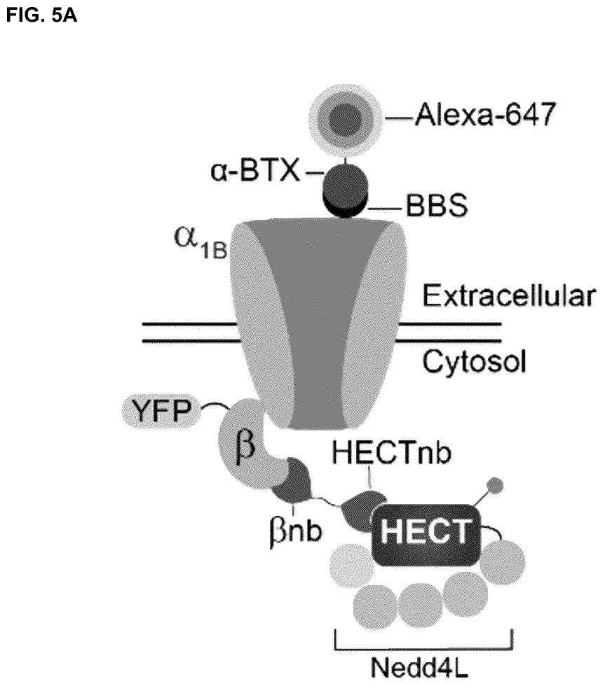

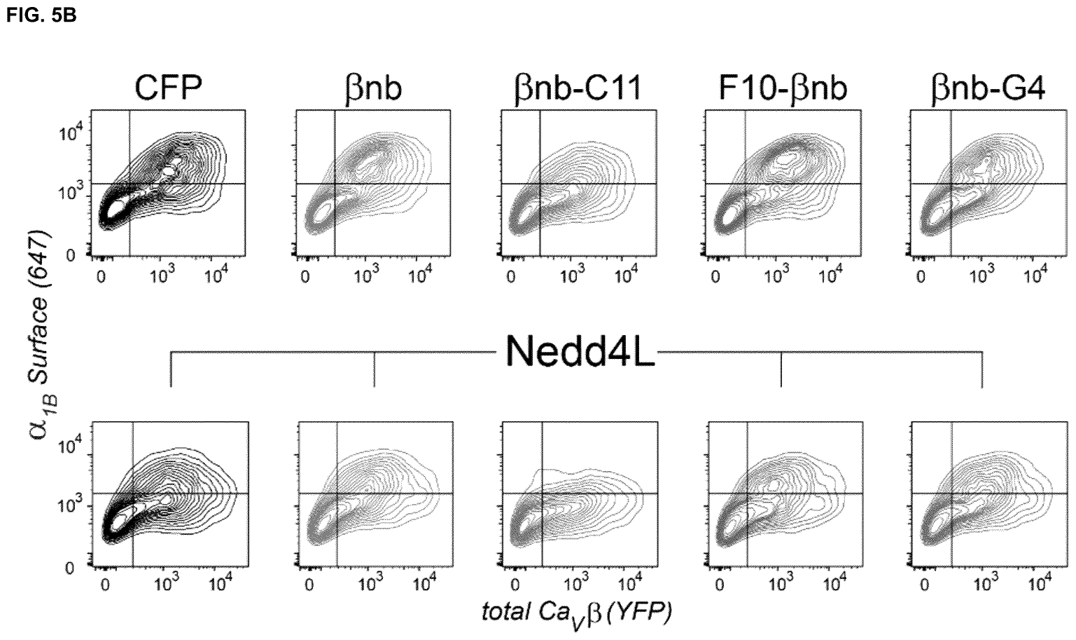

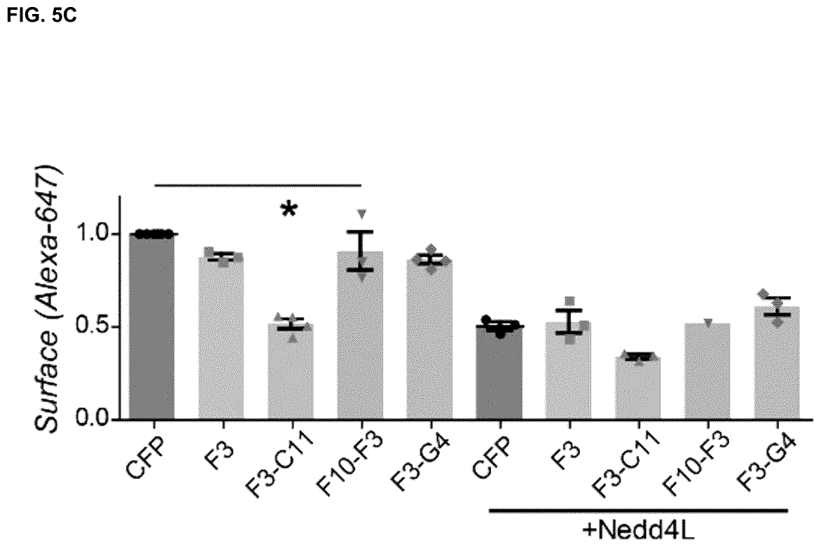

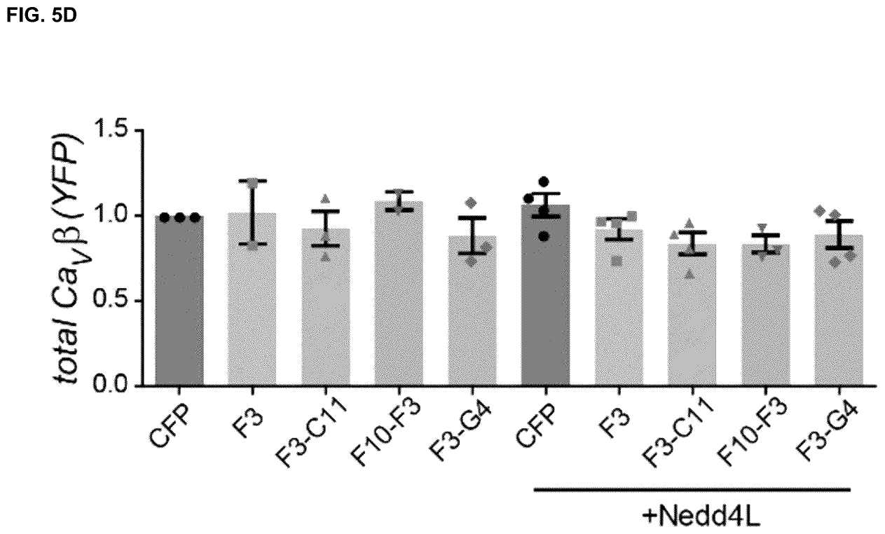

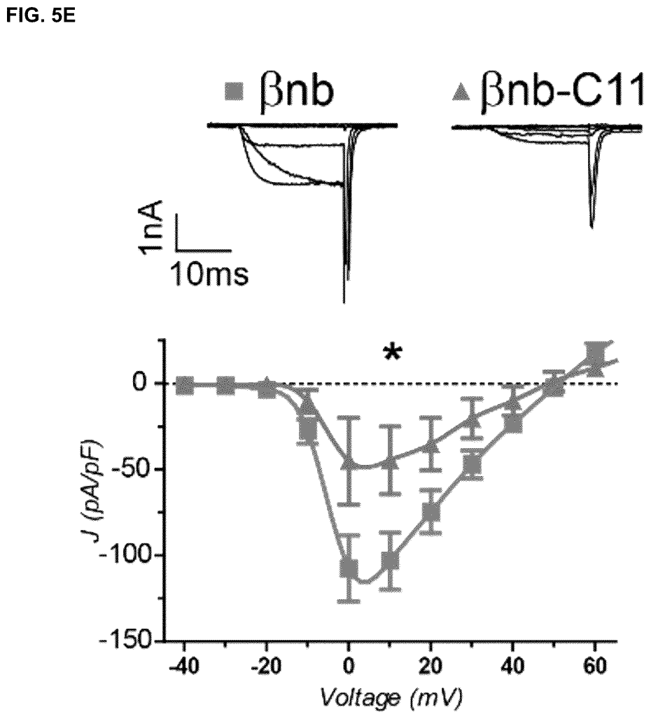

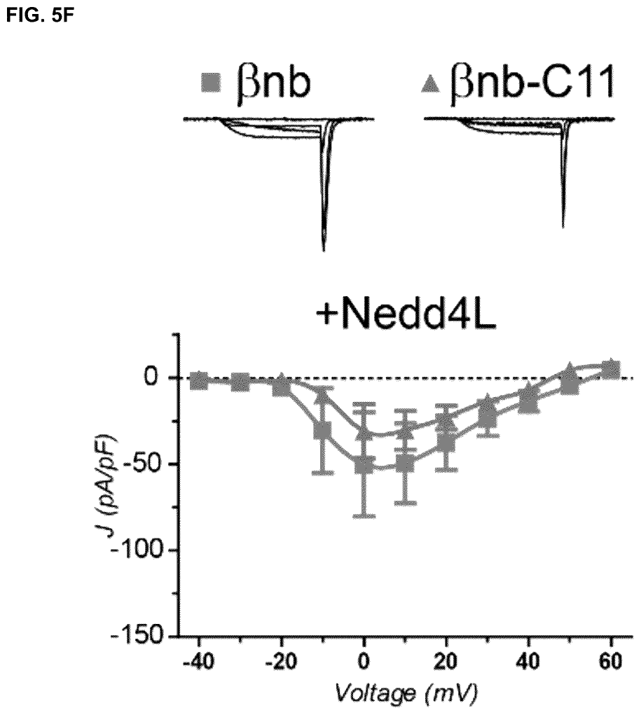

To assess the potential for our HECT_{Nedd4L} nanobodies to have a functional impact on HVACCs, we created Divaβ-Nedd4L constructs by tethering βnb and HECT_{Nedd4L} nanobodies with a (GGGGS)₂ linker, and placing this downstream of a CFP marker followed by the P2A self-cleaving peptide (Kim et al. 2011), enabling bicistronic expression of CFP and Divaβ-Nedd4L. We returned to our surface-labeling assay using cells transfected with BBS-α_{1B}, YFP-β₂ₐ, α₂β-1, and a particular Divaβ-Nedd4L; this enabled simultaneous measurements of surface levels of Caᵥ2.2 as well as total Caᵥβ levels (FIG. 5A). We first confirmed that βnb alone had no impact on channel trafficking, as compared to a control in which we co-transfected CFP. We then co-transfected the channel with one of three Divas and found that Divaβ-C11 had a significant effect on surface levels of Caᵥ2.2 (FIGS. 5B and 5C). Total Caᵥβ levels were unaffected by any Diva (FIGS. 5B and 5D), in agreement with our previous observations when we targeted the channel with the catalytic HECT_{Nedd4L} domain (Morgenstern et al. 2019). The above experiment assumes that there is endogenous Nedd4L expressed within HEK293 cells, as has been reported before (Cachemaille et al. 2012). Yet because our approach is dependent on Nedd4L expression, we repeated the experiment in the presence of exogenously expressed Nedd4L. Surprisingly, overexpression of Nedd4L reduced the surface density of Caᵥ2.2 nearly 50% while having no effect on total Caᵥβ levels (FIGS. 5B-5D); to our knowledge, this is the first report of Nedd4L regulating Caᵥ2.2. No Diva further altered surface levels of Caᵥ2.2 or total Caᵥβ levels when Nedd4L was overexpressed.

Finally, we confirmed the functional impact of Divaβ-C11 on reconstituted Caᵥ2.2 currents using whole-cell patch clamp measurements in HEK293 cells (FIGS. 5E-5F). Compared to control currents from cells transfected with βnb, Divaβ-C11 significantly reduced the maximal current density by approximately 50% (Jₚₑₐₖ at +10 mV=−103.3±16.67 pA pF⁻¹, N=11 for βnb; and −44.67±19.62 pA pF⁻¹, N=9 for Divaβ-C11, P<0.05, unpaired two-tailed Student's t test). Overexpression of Nedd4L had a similar effect as Divaβ-C11, reducing current density by 50% (Jₚₑₐₖ at +10 mV=−49.45±23.26 pA pF⁻¹, N=8), and Divaβ-C11 had no further significant impact on current density in the presence of over-expressed Nedd4L (Jₚₑₐₖ at +10 mV=−30.40±11.12 pA pF⁻¹, N=8). These results suggest that Nedd4L can target Caᵥ2.2, albeit the extent of this regulation is only seen upon over-expression of the ligase. Further, our results indicate that Divaβ-C11 is capable of enhancing this regulation at basal levels of Nedd4L to reduce Caᵥ2.2 function.

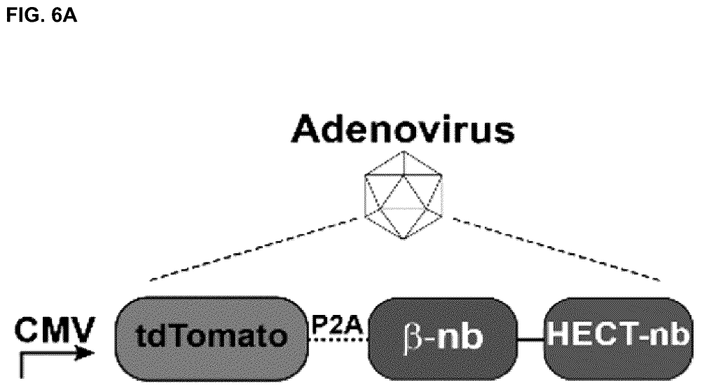

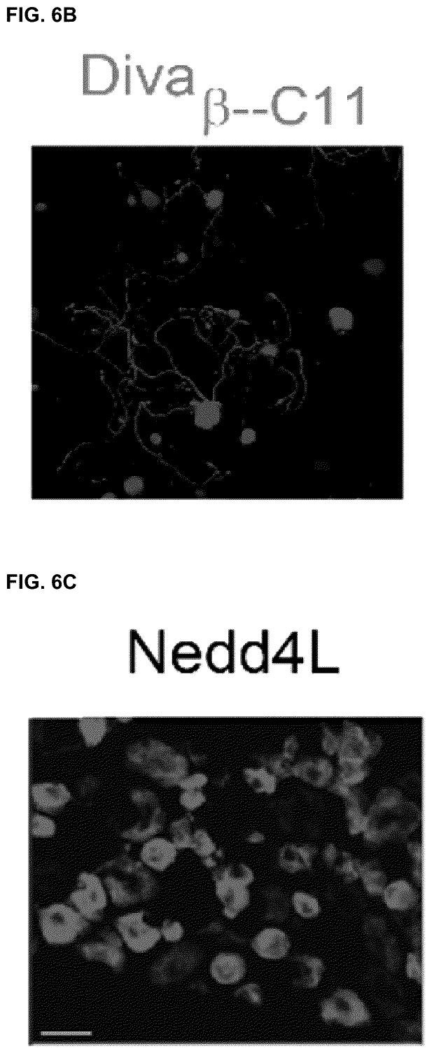

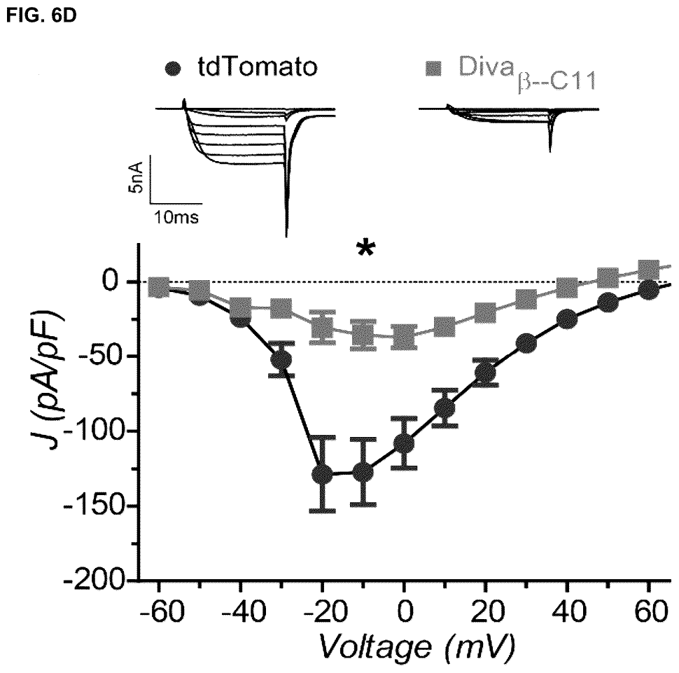

Given the effect of Divaβ-C11 on recombinant Caᵥ2.2 channels, we next tested whether we could use Divaβ-C11 to target endogenous HVACCs. One important caveat to this application is the requirement of endogenous Nedd4L expression in the tissue of interest. Intriguingly, both α_{1B} and Nedd4L are expressed in murine dorsal root ganglion (DRG) neurons, and each have been implicated in the pathophysiology of neuropathic pain (Schmidtko et al. 2010; Laedermann et al. 2013). Thus, we examined the potential for Divaβ-C11 to downregulate HVACCs by infecting cultured murine DRG neurons with adenovirus expressing either tdTomato or tdTomato-P2A-Divaβ-C11 (FIGS. 6A and 6B) and performed whole-cell patch clamp measurements in the presence of the selective t-type blocker Z944, in order to isolate HVACC currents (Zhao et al. 2019). As shown in FIG. 6D, Divaβ-C11 dramatically reduced HVACC currents (Jₚₑₐₖ at −10 mV=−127.3±21.67 pA pF⁻¹, N=8 for mCherry; −35.54±9.110 pA pF⁻¹, N=11 for Divaβ-C11. P<0.001, unpaired two-tailed Student's t-test). This powerful result demonstrates the potential for Divaβ-C11 to hijack endogenous Nedd4L for regulation of HVACCs.

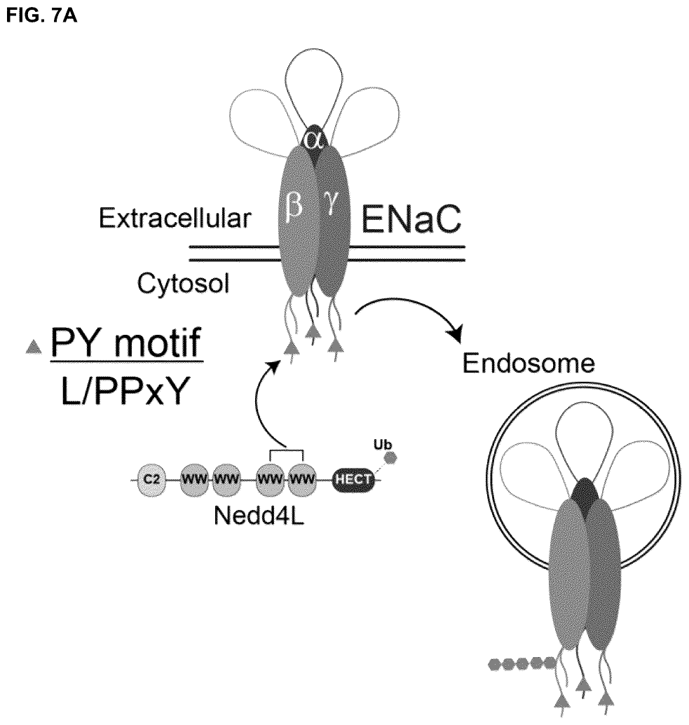

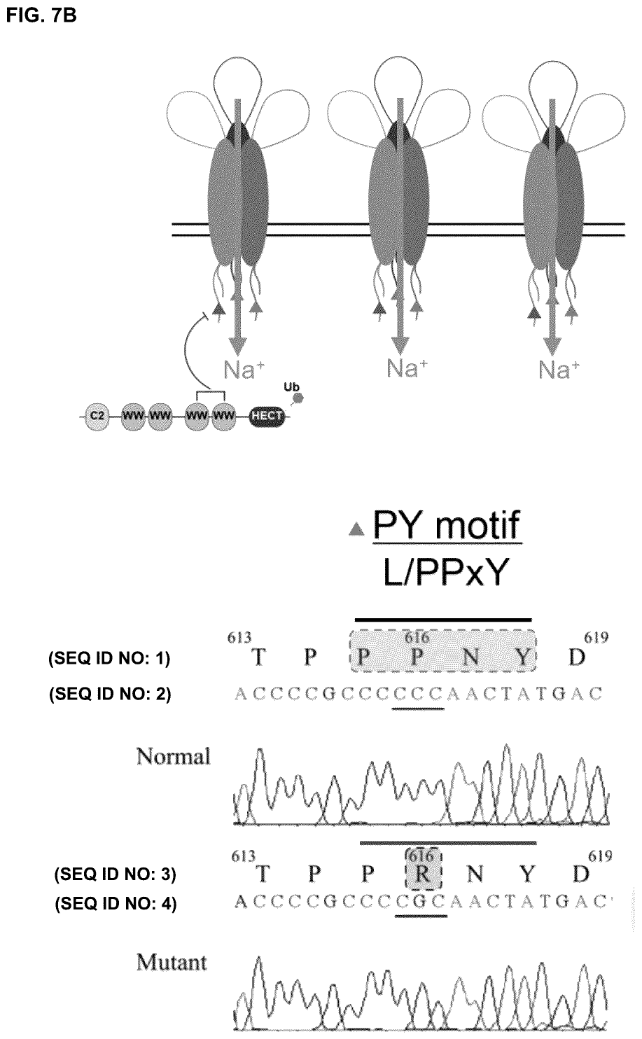

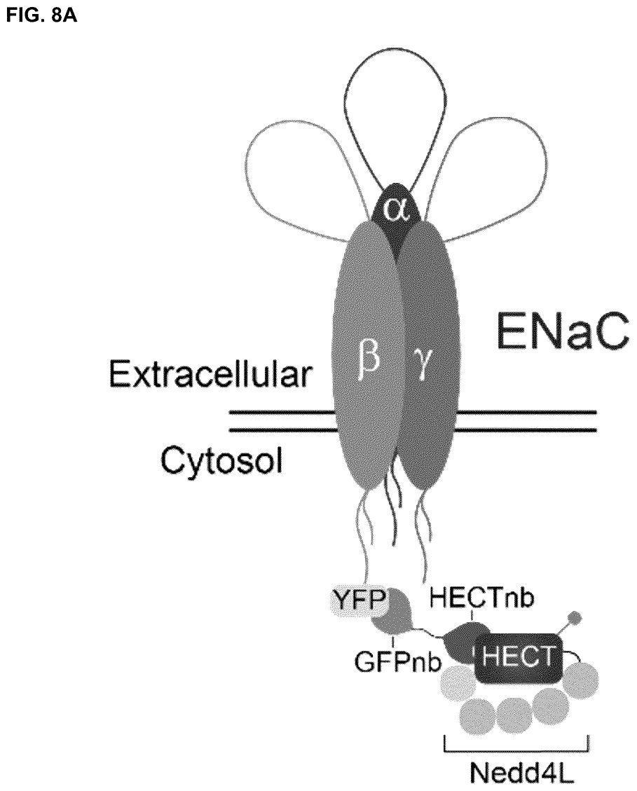

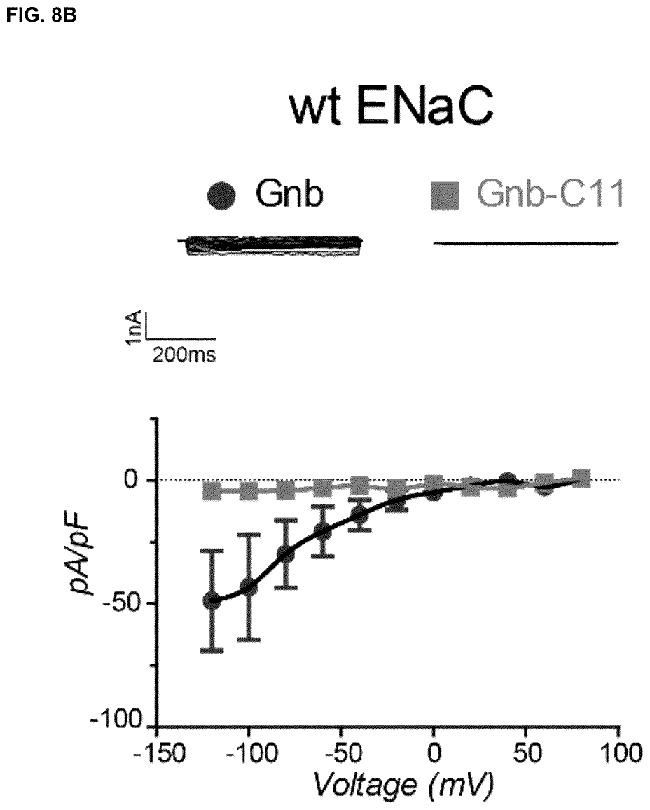

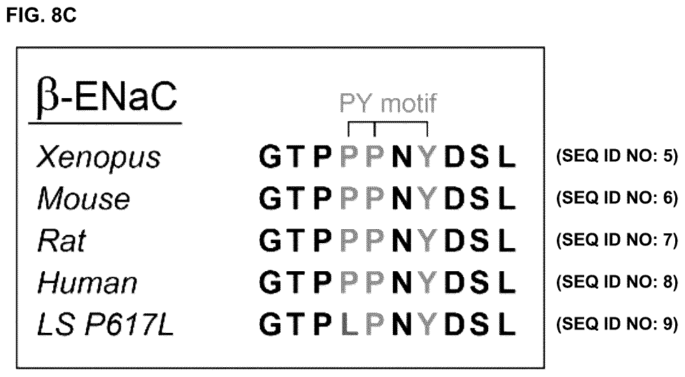

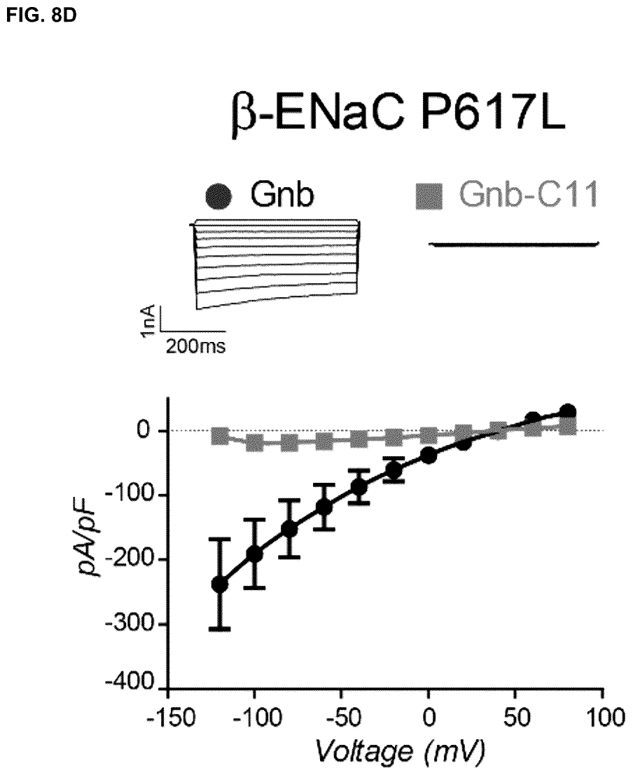

Lastly, we asked whether Divas could regulate other channels in a similar fashion by examining a well-established target of Nedd4L: the epithelial sodium channel (ENaC) (Raikwar and Thomas, 2008; Debonneville et al. 2001). ENaC is a trimeric transmembrane protein composed of homologous α, β, and γ subunits, that is permeable to Na⁺ ions and is critical for salt/water homeostasis (FIG. 7A) (Hanukoglu and Hanukoglu, 2016). We expressed α, γ, and YFP-tagged β-ENaC subunits in a 1:1 ratio in HEK293 cells and used a Diva comprised of the GFPnb tethered to nb.C11 (DivaGnb-C11) to target the β subunit of ENaC (FIG. 8A). Whole-cell patch clamp measurements from control cells transfected with ENaC and Gnb alone showed amiloride-sensitive ENaC currents, while ENaC currents from cells transfected with DivaGnb-C11 were essentially eliminated (FIG. 8B; Jₚₑₐₖ at −100 mV=−43.31±21.28 pA pF⁻¹, N=4 for Gnb; and −4.235±1.617 pA pF⁻¹, N=6 for DivaGnb-C11, P≤0.05, unpaired two-tailed Student's t-test). This result suggests that Divas may be used to target a variety of ion channels, but could Divas also be used to correct for dysregulation of a channel? In the case of ENaC, there are well-characterized mutations in the PY motifs of the β and γ subunits that prevent Nedd4L-mediated regulation of ENaC (Firsov et al. 1996; Lu et al. 2007); these mutations have been identified in patients to be the underlying cause of a distinct familial hypertension known as Liddle syndrome (FIG. 7B) (Hanukoglu and Hanukoglu, 2016). We postulated that Divas may be able to restore Nedd4L regulation of these mutant channels. To test this we again performed whole-cell patch clamp measurements in HEK293 cells, this time using a YFP-13-ENaC that contained a patient-derived mutation in its PY motif (P617L) (Rossi et al. 2008). In our control condition that expressed β-P617L ENaC+Gnb, we observed currents that were nearly 5-fold larger compared to wild type control conditions (FIG. 8D; Jₚₑₐₖ at −100 mV=−191.0±53.21 pA pF⁻¹, N=10). This observation provides evidence for the expression of endogenous Nedd4L in HEK293 cells, and also suggests that wild type ENaC is significantly downregulated by Nedd4L in this heterologous system. Strikingly, co-expression of DivaGFP-Nedd4L dramatically reduced β-P617L mutant ENaC current to below wild type levels (FIG. 8D; Jₚₑₐₖ at −100 mV=−18.48±7.33 pA pF⁻¹, N=9). By counteracting a mutation that is known to cause loss of Nedd4L regulation, this result provides evidence that Divas indeed work by recruiting Nedd4L for targeted regulation of the substrate. Altogether, the application of Divas to regulate reconstituted ENaC suggest that Divas may be a generalizable approach to regulate a number of ion channels.

### DISCUSSION

Here, we provide evidence that a PROTACS-inspired strategy, using nanobodies in place of small molecules, may be employed to recruit Nedd4L to regulate a target membrane protein. This may be surprising, given the extensive regulation of the catalytic HECT domain (Wiesner et al. 2007; Mari et al. 2014; Debonneville, 2001 #420; Chen, 2017; Bruce et al. 2008). Future work resolving the structural basis for nanobody C11-HECT_{Nedd4L} interaction will be critical in this regard. Regardless, the finding that nb.C11 is able to recruit a functional Nedd4L suggests that a small-molecule PROTACS approach may be possible. This possibility has broad relevance, as much research in the last twenty years has focused on manipulation of the ubiquitin system to regulate a protein of interest (Burslem and Crews 2020; Sakamoto et al. 2001), and is now beginning to make its heralded therapeutic impact (Mullard, 2019; Mullard, 2021).

Yet the current toolbox of small molecule ‘warheads’ that recruit an active E3 ubiquitin ligase are rather limited (Wu et al. 2020) and further, have not been shown to be effective at targeting multi-pass transmembrane proteins (Burslem et al. 2020). The HECT class of E3 ubiquitin ligases, which contains the Nedd4L family, have been shown to regulate multiple membrane proteins, including ion channels as well as GPCRs. A previous study that used a Halo-PROTAC approach to screen various E3 ligases for their utility in PROTACS concluded that recruitment of Nedd4L may not be feasible, as it did not show robust degradation of the GFP target (Ottis et al. 2017). However, in the case of membrane proteins such as ion channels, degradation may not be necessary for functional impairment; our previous work showed that a βnb-HECT_{Nedd4L} construct was able to redirect calcium channels from the plasma membrane to Rab7-containing late endosomes, resulting in functional inhibition (Morgenstern et al. 2019). Moreover, the ubiquitin code is complex, as different E3 ligases form distinct polyubiquitin chains that lead to different cellular outcomes; Nedd4L has been shown in vitro to add K63 polyubiquitin chains onto its substrate (Todaro et al. 2017), which are thought to be involved in protein sorting and endocytosis, while K48 polyubiquitin chains are primarily thought to direct substrates for proteasomal degradation (Komander and Rape, 2012). Thus, targeted recruitment of Nedd4L may not lead to proteolysis of a transmembrane protein of interest, but targeted ubiquitination may still produce desirable functional effects.

Yet there remains instances in which small molecules, including PROTACS, may be undesirable. In the case of HVACCs, small molecules and toxins are current and potential therapeutics for a variety of cardiovascular and neurological diseases (Zamponi et al. 2015). Yet the widespread distribution of small molecule HVACC blockers can lead to on-target/off-tissue side effects, narrowing their therapeutic window (Schmidtko et al. 2010). In these instances, GECCIs may be advantageous by providing long-lasting and tissue specific blockade of calcium channels. Here, we provide another potential layer of specificity to GECCIS, as Divas are dependent on expression of Nedd4L in the target tissue to regulate the target protein. Further, an exciting possibility is that the modular design of Divas suggests that this approach may be amenable to a heterodimerization strategy, in which the potency of Divas may be tuned with a small molecule.

### DOCUMENTS CITED

1 Ariel Bensimon, Mattia D. Pizzagalli, Felix Kartnig, Vojtech Dvorak, Patrick Essletzbichler, Georg E. Winter, Giulio Superti-Furga, Targeted Degradation of SLC Transporters Reveals Amenability of Multi-Pass Transmembrane Proteins to Ligand-Induced Proteolysis, Cell Chemical Biology, Volume 27, Issue 6, 2020, Pages 728-739.e9.
2. Bruce M C, Kanelis V, Fouladkou F, Debonneville A, Staub O, Rotin D. Regulation of Nedd4-2 self-ubiquitination and stability by a PY motif located within its HECT-domain. Biochem J. 2008 Oct. 1; 415(1):155-63.
3. Burslem G M, Crews C M. Proteolysis-Targeting Chimeras as Therapeutics and Tools for Biological Discovery. Cell. 2020 Apr. 2; 181(1):102-114.
4. Cachemaille M, Laedermann C J, Pertin M, Abriel H, Gosselin R D, Decosterd I. Neuronal expression of the ubiquitin ligase Nedd4-2 in rat dorsal root ganglia: modulation in the spared nerve injury model of neuropathic pain. Neuroscience. 2012 Dec. 27; 227:370-80.
5. Cani P D, Possemiers S, Van de Wiele T, Guiot Y, Everard A, Rottier O, Geurts L, Naslain D, Neyrinck A, Lambert D M, Muccioli G G, Delzenne N M. Changes in gut microbiota control inflammation in obese mice through a mechanism involving GLP-2-driven improvement of gut permeability. Gut. 2009 August; 58(8): 1091-103.
6. Catterall W A. Structure and regulation of voltage-gated Ca2+ channels. Annu Rev Cell Dev Biol. 2000; 16:521-55.
7 Chen Z. Mechanistic Insights into the Auto-Regulation of PTEN and Its Related HECT E3 Ligases. PhD dissertation submitted to Johns Hopkins University (2017).
8. Debonneville C, Flores S Y, Kamynina E, Plant P J, Tauxe C, Thomas M A, Münster C, Chraïbi A, Pratt J H, Horisberger J D, Pearce D, Loffing J, Staub O. Phosphorylation of Nedd4-2 by Sgk1 regulates epithelial Na(+) channel cell surface expression. EMBO J. 2001 Dec. 17; 20(24):7052-9.
9. Dolphin A C. Calcium channel auxiliary α25 and β subunits: trafficking and one step beyond. Nat Rev Neurosci. 2012 Jul. 18; 13(8):542-55.
10. Firsov D, Schild L, Gautschi I, Mérillat A M, Schneeberger E, Rossier B C. Cell surface expression of the epithelial Na channel and a mutant causing Liddle syndrome: a quantitative approach. Proc Natl Acad Sci USA. 1996 Dec. 24; 93(26):15370-5.
11. Hanukoglu I, Hanukoglu A. Epithelial sodium channel (ENaC) family: Phylogeny, structure-function, tissue distribution, and associated inherited diseases. Gene. 2016 Apr. 1; 579(2):95-132.
12. Hayer, A., and Bhalla, U. S. (2005). Molecular switches at the synapse emerge from receptor and kinase traffic. PLoS Comput. Biol. 1, e20.
13. Hicke L. A new ticket for entry into budding vesicles-ubiquitin. Cell. 2001 Sep. 7; 106(5):527-30.
14. Kim J H, Lee S R, Li L H, Park H J, Park J H, Lee K Y, Kim M K, Shin B A, Choi S Y. High cleavage efficiency of a 2A peptide derived from porcine teschovirus-1 in human cell lines, zebrafish and mice. PLoS One. 2011; 6(4):e18556.
15. Komander D, Rape M. The ubiquitin code. Annu Rev Biochem. 2012; 81:203-29.
16. Kubala M H, Kovtun O, Alexandrov K, Collins B M. Structural and thermodynamic analysis of the GFP:GFP-nanobody complex. Protein Sci. 2010 December; 19(12):2389-401.
17. Laedermann C J, Cachemaille M, Kirschmann G, Pertin M, Gosselin R D, Chang I, Albesa M, Towne C, Schneider B L, Kellenberger S, Abriel H, Decosterd I. Dysregulation of voltage-gated sodium channels by ubiquitin ligase NEDD4-2 in neuropathic pain. J Clin Invest. 2013 July; 123(7):3002-13.
18. Lai, A. C., Toure, M., Hellerschmied, D., Salami, J., Jaime-Figueroa, S., Ko, E., Hines, J. and Crews, C. M. (2016), Modular PROTAC Design for the Degradation of Oncogenic BCR-ABL. Angew. Chem. Int. Ed., 55: 807-810.
19. Lu C, Pribanic S, Debonneville A, Jiang C, Rotin D. The PY motif of ENaC, mutated in Liddle syndrome, regulates channel internalization, sorting and mobilization from subapical pool. Traffic. 2007 September; 8(9):1246-64.
20. MacGurn J A, Hsu P C, Emr S D. Ubiquitin and membrane protein turnover: from cradle to grave. Annu Rev Biochem. 2012; 81:231-59.
21. Mari S, Ruetalo N, Maspero E, Stoffregen M C, Pasqualato S, Polo S, Wiesner S. Structural and functional framework for the autoinhibition of Nedd4-family ubiquitin ligases. Structure. 2014 Nov. 4; 22(11):1639-49.
22. McMahon C, Baier A S, Pascolutti R, Wegrecki M, Zheng S, Ong J X, Erlandson S C, Hilger D, Rasmussen S G F, Ring A M, Manglik A, Kruse A C. Yeast surface display platform for rapid discovery of conformationally selective nanobodies. Nat Struct Mol Biol. 2018 March; 25(3):289-296.
23. Morgenstern T J and Colecraft H M. Controlling ion channel trafficking by targeted ubiquitination and deubiquitination. Methods Enzymol. 2021; 654:139-167.
24. Morgenstern T J, Park J, Fan Q R, Colecraft H M. A potent voltage-gated calcium channel inhibitor engineered from a nanobody targeted to auxiliary Ca V β subunits. eLife 2019; 8:e49253.
25. Mullard A. First targeted protein degrader hits the clinic. Nat Rev Drug Discov. 2019 Mar. 6.
26. Mullard A. Targeted protein degraders crowd into the clinic. Nat Rev Drug Discov. 2021 April; 20(4):247-250.
27. Ottis P, Toure M, Cromm P M, Ko E, Gustafson J L, Crews C M. Assessing Different E3 Ligases for Small Molecule Induced Protein Ubiquitination and Degradation. ACS Chem Biol. 2017 Oct. 20; 12(10):2570-2578.
28. Raikwar N S, Thomas C P. Nedd4-2 isoforms ubiquitinate individual epithelial sodium channel subunits and reduce surface expression and function of the epithelial sodium channel. Am J Physiol Renal Physiol. 2008 May; 294(5):F1157-65.
29. Rivas S, Hanif K, Chakouri N, Ben-Johny M. Probing ion channel macromolecular interactions using fluorescence resonance energy transfer. Methods Enzymol. 2021; 653:319-347.
30. Rossi E, Farnetti E, Debonneville A, Nicoli D, Grasselli C, Regolisti G, Negro A, Perazzoli F, Casali B, Mantero F, Staub O. Liddle's syndrome caused by a novel missense mutation (P617L) of the epithelial sodium channel beta subunit. J Hypertens. 2008 May; 26(5):921-7.
31. Sakamoto K M, Kim K B, Kumagai A, Mercurio F, Crews C M, Deshaies R J. Protacs: chimeric molecules that target proteins to the Skp1-Cullin-F box complex for ubiquitination and degradation. Proc Natl Acad Sci USA. 2001 Jul. 17; 98(15):8554-9.
32. Schmidtko A, Lötsch J, Freynhagen R, Geisslinger G. Ziconotide for treatment of severe chronic pain. Lancet. 2010 May 1; 375(9725):1569-77.
33. Todaro D R, Augustus-Wallace A C, Klein J M, Haas A L. The mechanism of neural precursor cell expressed developmentally down-regulated 4-2 (Nedd4-2)/NEDD4L-catalyzed polyubiquitin chain assembly. J Biol Chem. 2017 Nov. 24; 292(47): 19521-19536.
34. Wiesner S, Ogunjimi A A, Wang H R, Rotin D, Sicheri F, Wrana J L, Forman-Kay J D. Autoinhibition of the HECT-type ubiquitin ligase Smurf2 through its C2 domain. Cell. 2007 Aug. 24; 130(4):651-62.
35. Wu J, Xia L, Yao X, Yu X, Tumas K C, Sun W, Cheng Y, He X, Peng Y C, Singh B K, Zhang C, Qi C F, Bolland S, Best S M, Gowda C, Huang R, Myers T G, Long C A, Wang R F, Su X Z. The E3 ubiquitin ligase MARCH1 regulates antimalaria immunity through interferon signaling and T cell activation. Proc Natl Acad Sci USA. 2020 Jul. 14; 117(28):16567-16578.
36. Xu J, Wang W, Clark C C, Brighton C T. Signal transduction in electrically stimulated articular chondrocytes involves translocation of extracellular calcium through voltage-gated channels. Osteoarthritis Cartilage. 2009 March; 17(3):397-405.
37. Yau R G, Doerner K, Castellanos E R, Haakonsen D L, Werner A, Wang N, Yang X W, Martinez-Martin N, Matsumoto M L, Dixit V M, Rape M. Assembly and Function of Heterotypic Ubiquitin Chains in Cell-Cycle and Protein Quality Control. Cell. 2017 Nov. 2; 171(4):918-933.e20.
38. Zamponi G W, Striessnig J, Koschak A, Dolphin A C. The Physiology, Pathology, and Pharmacology of Voltage-Gated Calcium Channels and Their Future Therapeutic Potential. Pharmacol Rev. 2015 October; 67(4):821-70.
39. Zhao Y, Huang G, Wu Q, Wu K, Li R, Lei J, Pan X, Yan N. Cryo-EM structures of apo and antagonist-bound human Caᵥ3.1. Nature. 2019 December; 576(7787):492-497.

All patents, patent applications, and publications cited above are incorporated herein by reference in their entirety as if recited in full herein.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

What is claimed is:

1. A chimeric divalent molecule comprising:
(a) an E3 ubiquitin ligase binder;
(b) a target binder; and
(c) a variable linker between the E3 ubiquitin ligase binder and the target binder.

2. The chimeric divalent molecule of claim 1, wherein the E3 ubiquitin ligase binder and the target binder are both nanobodies.

3. The chimeric divalent molecule of claim 1, wherein the E3 ubiquitin ligase is a HECT domain E3 ubiquitin ligase.

4. The chimeric divalent molecule of claim 3, wherein the HECT domain E3 ubiquitin ligase is Nedd4L.

5. The chimeric divalent molecule of claim 4, wherein the E3 ubiquitin ligase binder recruits an active Nedd4L to the target.

6. The chimeric divalent molecule of claim 4, wherein the E3 ubiquitin ligase binder selectively blocks the action of Nedd4L on the target.

7. The chimeric divalent molecule of claim 1, wherein the target is a protein selected from ion channels, G protein-coupled receptors (GPCRs), cystic fibrosis transmembrane conductance regulator (CFTR), transporters, and kinases.

8. The chimeric divalent molecule of claim 1, wherein the target is an ion channel selected from the voltage-gated calcium channel, CaV2.2, the voltage-gated potassium channel, KCNQ1, and the epithelial sodium channel, ENaC.

9. The chimeric divalent molecule of claim 1, wherein the target is a high voltage-activated calcium channel (HVACC).

10. The chimeric divalent molecule of claim 9, wherein the HVACC is an N-type calcium channel.

11. The chimeric divalent molecule of claim 9, wherein the target is the auxiliary CaVβ subunit of the HVACC.

12. A method for treating or ameliorating the effects of a disease in a subject, comprising administering to the subject an effective amount of a chimeric divalent molecule according to claim 1.

13. The method of claim 12, wherein the disease is selected from the group consisting of an inherited ion channelopathy, a cancer, a cardiovascular condition, an infectious disease, and a metabolic disease.

14. The method of claim 13, wherein the inherited ion channelopathy is selected from the group consisting of epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus.

15. The method of claim 12, wherein the disease is selected from the group consisting of hypertension, Liddle syndrome, cardiac arrhythmias, diabetes, Parkinson's disease, and chronic pain.

16. The method of claim 12, wherein the disease is chronic pain.


SEQUENCE LISTING (9 sequences)

SEQ ID NO: 1 - protein, 7 aa, Homo sapiens
TPPPNYD

SEQ ID NO: 2 - DNA, 21 bp, Homo sapiens
  source 1..21: mol_type = genomic DNA
accccgccccccaactatgac

SEQ ID NO: 3 - protein, 7 aa, Homo sapiens
TPPRNYD

SEQ ID NO: 4 - DNA, 21 bp, Homo sapiens
  source 1..21: mol_type = genomic DNA
accccgccccgcaactatgac

SEQ ID NO: 5 - protein, 10 aa, Xenopus borealis
GTPPPNYDSL

SEQ ID NO: 6 - protein, 10 aa, Mus abbotti
GTPPPNYDSL

SEQ ID NO: 7 - protein, 10 aa, Rattus exulans
GTPPPNYDSL

SEQ ID NO: 8 - protein, 10 aa, Homo sapiens
GTPPPNYDSL

SEQ ID NO: 9 - protein, 10 aa, Homo sapiens
GTPLPNYDSL